(19) United States
(12) Patent Application Publication
(10) Pub. No.: US 2023/0254010 A1
(43) Pub. Date: Aug. 10, 2023

(54) SYSTEMS FOR IONIC COMMUNICATION IN ELECTROLYTE

(71) Applicants: Dion Khodagholy, Cresskill, NJ (US); Zifang Zhao, New York, NY (US)

(72) Inventors: Dion Khodagholy, Cresskill, NJ (US); Zifang Zhao, New York, NY (US)

(21) Appl. No.: 18/106,467

(22) Filed: Feb. 6, 2023

Related U.S. Application Data
(60) Provisional application No. 63/307,152, filed on Feb. 6, 2022.

(51) Int. Cl. H04B 5/00; A61B 5/00

(52) U.S. Cl. CPC H04B 5/0012; A61B 5/0031

(57) ABSTRACT

System for ionic communication in an electrolyte are provided, the systems including a transmitter; a first plurality of electrodes coupled to the transmitter and in contact with an electrolyte; a receiver; and a second plurality of electrodes coupled to the receiver and in contact with the electrolyte, wherein the transmitter is configured to transmit at least one signal to the receiver by manipulating ions in the electrolyte using the first plurality of electrodes. In some of these systems, the transmitter and the first plurality of electrodes are configured to be placed inside a body comprising the electrolyte. In some of these systems, the first plurality of electrodes consists of two electrodes. In some of these systems, the first plurality of electrodes includes at least three electrodes and the at least one signal is a plurality of signals.

### CROSS-REFERENCE TO RELATED APPLICATION

This application claims the benefit of U.S. Provisional Patent Application No. 63/307,152, filed Feb. 6, 2022, which is hereby incorporated by reference herein in its entirety.

### STATEMENT REGARDING GOVERNMENT FUNDED RESEARCH

This invention was made with government support under grants EY032381, NS118091, and NS108923 awarded by the National Institutes of Health and grants 1944415 and 2027135 awarded by the National Science Foundation. The government has certain rights in the invention.

### BACKGROUND

Implanted bioelectronic devices are increasingly being used to monitor and treat disease. Signal transmission from an implanted device to external electronics is a major challenge for safe, effective, long-term use.

Physiologic signals are robustly transmitted by cables due to their simplicity and high data rate capacity, but this approach requires permanent tissue traversing components that limit their use in chronic applications.

Wireless data transmission from implanted devices has been accomplished using radio frequency (RF) and ultrasound-based communication. The complex, high-power consumption, non-biocompatible, and rigid RF electronic components combined with the high ionic conductivity of biological tissue place severe restrictions on signal transmission capabilities of RF communication. As a result, the majority of RF-based systems require tissue extruding components that interface with a transmitter placed outside the body. Although ultrasound has better tissue penetration than RF, communication is strongly dependent on the coupling factor between the transmitter and receiver, allowing tissue inhomogeneity and mechanical movements to introduce instability.

Optical methods have high power consumption, and are limited by light scattering within tissue.

Accordingly, new mechanisms for communicating data from implanted devices are desirable.

### SUMMARY

In some embodiments, systems for ionic communication in electrolyte are provided, the systems comprising: a transmitter; a first plurality of electrodes coupled to the transmitter and in contact with the electrolyte; a receiver; and a second plurality of electrodes coupled to the receiver and in contact with the electrolyte, wherein the transmitter is configured to transmit at least one signal to the receiver by manipulating ions in the electrolyte using the first plurality of electrodes. In some of these embodiments, the transmitter and the first plurality of electrodes are configured to be placed inside a body comprising the electrolyte. In some of these embodiments, the first plurality of electrodes consists of two electrodes. In some of these embodiments, the first plurality of electrodes includes at least three electrodes and the at least one signal is a plurality of signals. In some of these embodiments, a voltage of the signal is less than 200 millivolts. In some of these embodiments, a frequency of the signal is between 10 kHz to 10 MHz. In some of these embodiments, the electrolyte is a human body. In some of these embodiments, the at least one signal is transmitted from inside a body to outside the body. In some of these embodiments, the first plurality of electrodes includes at least one gold electrode. In some of these embodiments, the first plurality of electrodes includes at least one conducting polymer electrode. In some of these embodiments, the first plurality of electrodes are arranged in a honeycomb configuration.

In some embodiments, systems for ionic communication in electrolyte are provided, the systems comprising: a transmitter; and a first plurality of electrodes coupled to the transmitter and in contact with the electrolyte, wherein the transmitter is configured to transmit at least one signal by manipulating ions in the electrolyte using the first plurality of electrodes. In some of these embodiments, the transmitter and the first plurality of electrodes are configured to be placed inside a body comprising the electrolyte. In some of these embodiments, the first plurality of electrodes consists of two electrodes. In some of these embodiments, the first plurality of electrodes includes at least three electrodes and the at least one signal is a plurality of signals.

In some embodiments, systems for ionic communication in electrolyte are provided, the systems comprising: a receiver; and a first plurality of electrodes coupled to the receiver and in contact with the electrolyte, wherein the receiver is configured to receive at least one signal in response to ions in the electrolyte being manipulated. In some of these embodiments, the receiver and the first plurality of electrodes are configured to be placed on top of skin of a body comprising the electrolyte. In some of these embodiments, the first plurality of electrodes consists of two electrodes. In some of these embodiments, the first plurality of electrodes includes at least three electrodes and the at least one signal is a plurality of signals.

### BRIEF DESCRIPTION OF THE DRAWINGS

FIG. 1 is an example of ionic communication in accordance with some embodiments.

FIG. 2 is an example of a graph of response versus frequency in accordance with some embodiments.

FIGS. 3A and 3B are examples of graphs of impedance versus frequency and corner frequency versus size in accordance with some embodiments.

FIG. 4 is an example of an illustration of transmitter and receiver electrode geometry in accordance with some embodiments.

FIG. 5 is an example of a graph of response versus W×L/D (as shown in FIG. 4) in accordance with some embodiments.

FIG. 6 is an example of a graph of transmission distances versus W×L (as shown in FIG. 4) for different transmission voltages in accordance with some embodiments.

FIG. 7 is an example of an illustration of an electrode arrangement in accordance with some embodiments.

FIG. 8 is an example of an illustration showing an offset between receiver and transmitter electrodes in accordance with some embodiments.

FIG. 9 is an example of a block diagram of a transmitter in accordance with some embodiments.

FIG. 10 is an example of a schematic showing an electrophysiology amplifier that can be used in accordance with some embodiments.

FIG. 11 is an example of a schematic showing an accelerometer that can be used in a transmitter in accordance with some embodiments.

FIG. 12 is an example of a schematic showing a magnetic switch that can be used in a transmitter in accordance with some embodiments.

FIG. 13 is an example of a schematic showing a hardware processor that can be used in a transmitter in accordance with some embodiments.

FIG. 14 is an example of a schematic showing a crystal that can be used in a transmitter in accordance with some embodiments.

FIG. 15 is an example of a schematic showing voltage regulators that can be used in a transmitter in accordance with some embodiments.

FIG. 16 is an example of a schematic showing an attenuator and filter that can be used in a transmitter in accordance with some embodiments.

FIG. 17 is an example of a block diagram of a receiver in accordance with some embodiments.

FIG. 18 is an example of a schematic showing an example of how components of FIG. 17 can be implemented in accordance with some embodiments.

### DETAILED DESCRIPTION

In accordance with some embodiments, mechanisms (which can include systems, methods, and media) for ionic communication (IC) in electrolyte are provided.

In some embodiments, these mechanisms manipulate ions in electrolyte in biologic tissue to propagate MHz-range signals. In some embodiments, IC operates by generating and sensing stored electrical potential energy within polarizable media in a frequency-dependent manner. In some embodiments, geometric properties that govern IC transmission depth and transmission radius can be controlled to permit multi-line parallel communication. In some embodiments, IC can be used for real-time transmission of multi-channel local field potential (LFP) and neural spiking data, with data quality sufficient for clustering of individual neuronal action potentials. In some embodiments, IC can create a high-speed, low-power link between fully implanted electronics and external electronics with the potential to enhance the safety and efficiency of a wide range of bioelectronic devices.

In some embodiments, IC uses a polarizable medium to store/sense potential energy and transmit data. Compared to conventional radio-frequency (RF) communications, IC does not use propagating RF waves generated by an antenna. Instead, it uses multiple implanted electrodes of a transmitter to manipulate ions in an electrolyte (e.g., polarize tissue) and another set of multiple electrodes of a receiver to sense the potential energy, in some embodiments.

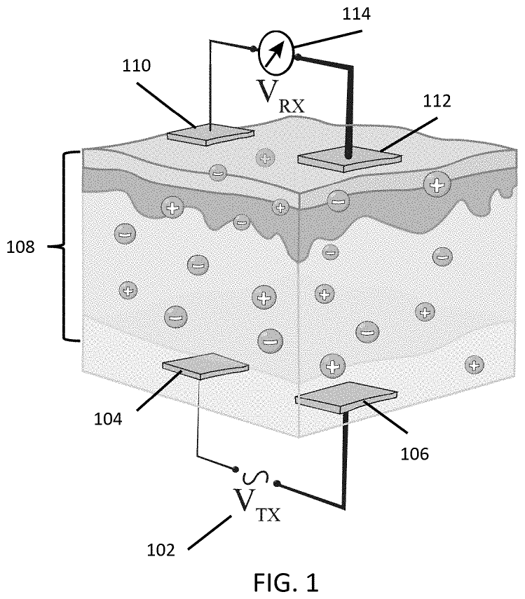

Turning to FIG. 1, an example of IC in accordance with embodiment some embodiments is illustrated. As shown, two pairs of electrodes 104,106 and 110,112 are placed parallel to each other across tissue 108 containing an electrolyte. One pair of electrodes 104,106 serve as transmitter electrodes and one pair of electrodes 110,112 serve as receiver electrodes. A transmitted signal V_{TX} 102 is generated by a transmitter (not shown) and provided to the transmitter electrodes. The transmitter electrodes 104,106 manipulate ions in the electrolyte (e.g., including polarizing the electrolyte in some embodiments), and this manipulations is sensed by receiver electrodes 110,112 and a receiver (not shown) connected thereto as received signal V_{RX} 114.

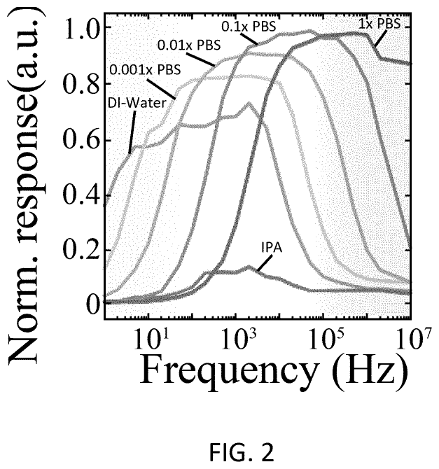

FIG. 2 shows an example of frequency response measurements for transmission of a constant amplitude frequency sweep signal from 1 Hz-10 MHz from a pair of transmitter electrodes to a pair of receiver electrodes in media with varying ion concentrations of phosphate buffer saline (PBS) (10⁻³, 10⁻², 10⁻¹ and 1×, PBS), de-ionized-water (DI-water), and isopropyl alcohol (IPA), in some embodiments. As can be seen, each frequency response curve is characterized by an inverted U pattern, with increasing response between 1 Hz-1 kHz, an intervening plateau, and decreasing response above 10 kHz-100 MHz. Increasing ion concentration shifts the curve to the right, resulting in higher response at higher frequencies relative to lower ion concentrations, in some embodiments.

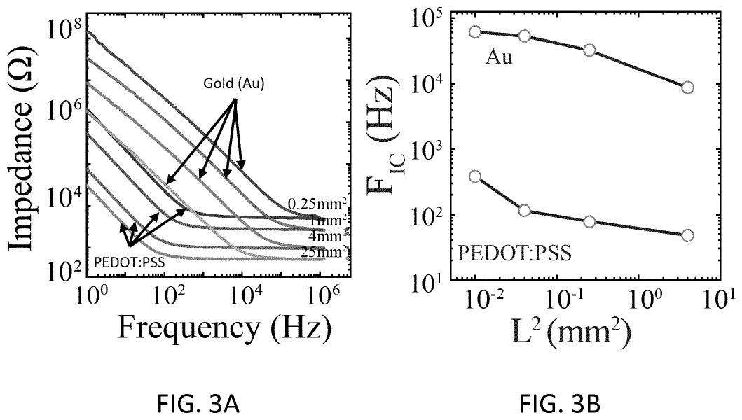

FIGS. 3A and 3B respectively show example graphs of impedance versus frequency and corner frequency (F_{IC}) versus size for gold (Au) electrode pairs and electrode pairs formed from a conducting polymer (Poly-3,4-ethylenedioxythiophene-polystyrenesulfonate, PEDOT:PSS) in accordance with some embodiments. As illustrated in FIG. 3A, in some embodiments, larger electrode size is associated with decreased impedance across the frequency spectrum. As illustrated in FIG. 3B, in some embodiments, using conducting polymer as transmitter and receiver electrode material can extend the bandwidth for IC by two orders of magnitude for any given electrode size.

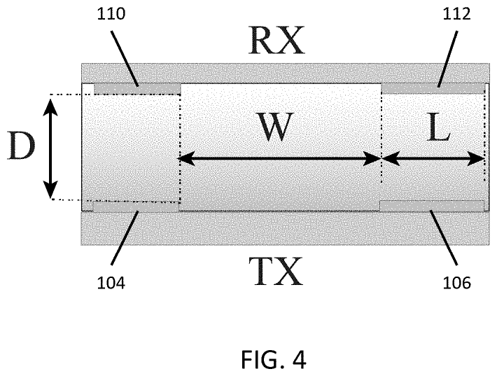

FIG. 4 shows an example illustration of receiver electrodes 110 and 112 and transmitter electrodes 104 and 106 separated by a medium, such as an electrolyte, in some embodiments. As shown, receiver electrodes 110 and 112 are separated by a distance W and transmitter electrodes 104 and 106 are separated by the distance W. Electrodes 110 and 104 are separated by a distance D and electrodes 112 and 106 are separated by a distance D. And, each electrode 104, 106, 110, and 112 has a length L.

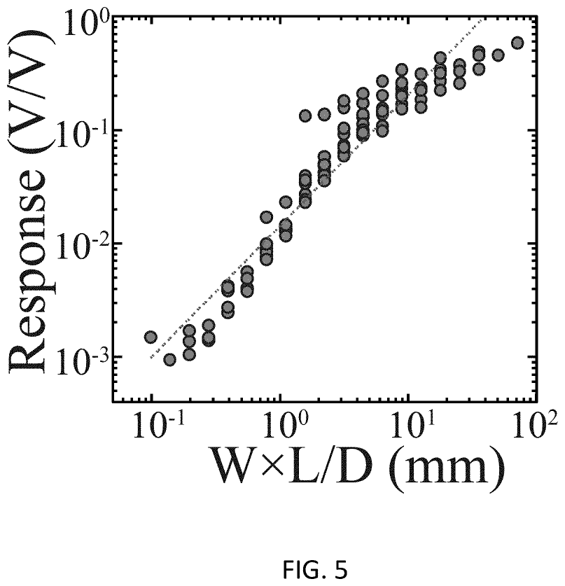

Turning to FIG. 5, an example graph showing frequency response versus W×L/D (as described above in connection with FIG. 4) is illustrated in accordance with embodiment some embodiments. As shown, IC frequency response is linearly and independently correlated with each measure, increasing proportionally with L and W, but decreasing with D, in some embodiments. As such, any combination of L and W that results in the same multiplicative product can be associated with the same D, in some embodiments.

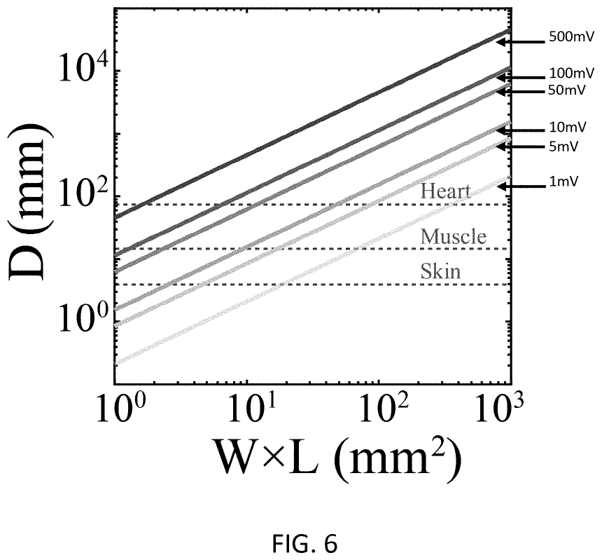

FIG. 6 shows an example graph of distance D versus W×L (as described above in connection with FIG. 4) for different transmitter voltages (i.e., 1 mV, 5 mV, 10 mV, 50 mV, 100 mV, and 500 mV) in accordance with some embodiments. As can be seen, a range of IC electrode geometries compatible with implantation are capable of communicating across distances required to target a variety of tissue, including the human heart, human muscle, and human skin, in some embodiments.

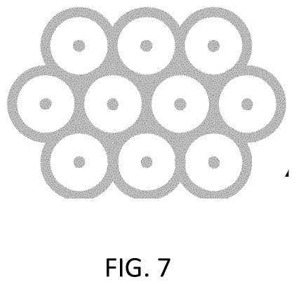

FIG. 7 shows an example arrangement of electrodes that can be used in some embodiments. As illustrated, ten electrodes can be used at the same time to realize multiple parallel lines of data communication, in some embodiments. Also as illustrated, the electrodes can be arranged in a honeycomb arrangement, in some embodiments. In some embodiments, with parallel lines, the rate of data communication can be dramatically increased without necessitating higher operating frequency.

Any suitable number of electrodes can be used in some embodiments. For example, in some embodiments, three electrodes can be used, in which two of the electrodes share a common reference electrode. As another example, in some embodiments, four electrodes can be used, and two of the electrodes can be referenced to a corresponding other two of the electrodes, but share no common reference.

Any suitable shape of electrodes can be used in some embodiments. For example, as shown in FIG. 1, electrodes can be square or rectangular in some embodiments. As another example, as shown in FIG. 7, electrodes can be circular in some embodiments. As still other examples, electrodes can be triangular, hexagonal, octagonal, trapezoidal, or any other suitable shape, in some embodiments.

Any suitable arrangement of electrodes can be used in some embodiments. For example, as shown in FIG. 1, two or more electrodes can be places along a line in some embodiments. As another example, as shown in FIG. 7, electrodes can be arranged in a honeycomb configuration in some embodiments. As still another example, electrodes can be placed in a grid arrangement in some embodiments.

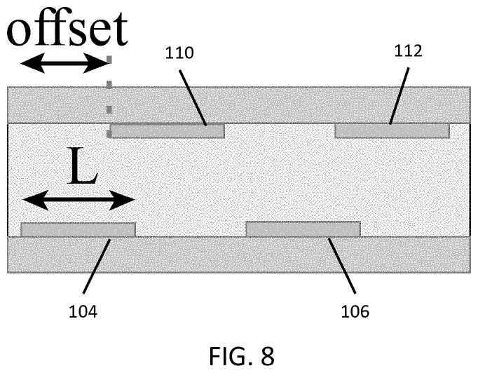

As shown in FIG. 8, receiver electrodes 110, 112 can be offset from transmitter electrodes 104,106, in some embodiments. In some embodiments, the most effective communication (highest response across frequency range) can be observed when receiver electrodes 110,112 and transmitter electrodes 104,106 are directly aligned—i.e., have zero offset. In some embodiments, upon reaching misalignments larger than the electrode geometry (L) a dramatic decay in response can be observed. Because the response can drop sharply at the boundary of transmitter and receiver electrode overlap, a densely packed conformable array of IC transmitters can be created in a co-axial format without introducing cross-talk between the independent transmitters, in some embodiments.

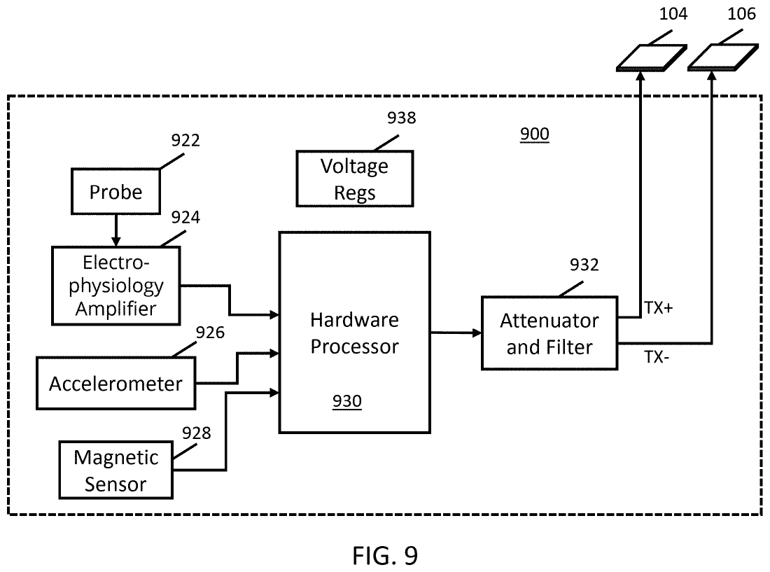

Turning to FIG. 9, an example 900 of a transmitter that can be used to drive transmitter electrodes 104,106 of FIG. 1 in accordance with some embodiments is shown. As illustrated, transmitter 900 includes a probe 922, an electrophysiology amplifier 924, an accelerometer 926, a magnetic sensor 928, a hardware processor 930, an attenuator and filter 932, and voltage regulators 938. In some embodiments, transmitter 900 along with electrodes 104 and 106 can be fully implanted in the body of a person or an animal being monitored, and the transmitter can use IC to communicate data regarding the person or animal being monitored to a receiver and electrodes 110 and 112 on the skin of the body of the person or animal being monitored.

In some embodiments, transmitter 900 can operate as follows. Initially, the transmitter can be in a low-power or sleep state. Next, in response to a magnet being placed near magnetic sensor 928 and the sensor detecting that magnet, the transmitter can turn on. Then, the probe and electrophysiology amplifier can and/or the accelerometer can detect activity and convey corresponding data to the hardware processor. The hardware processor can perform any suitable processing on the data and encode the data into a charge balanced protocol. A UART output of the hardware processor can then provide transmission signal based on the charge balanced protocol to attenuator and filter 932 which can attenuate the transmission signals (e.g., 100 times), high-pass filter the signals (e.g., f_{C}=100 kHz), and send the signals to the transmitter electrodes. The signal can then be conveyed to a receiver via IC as described herein.

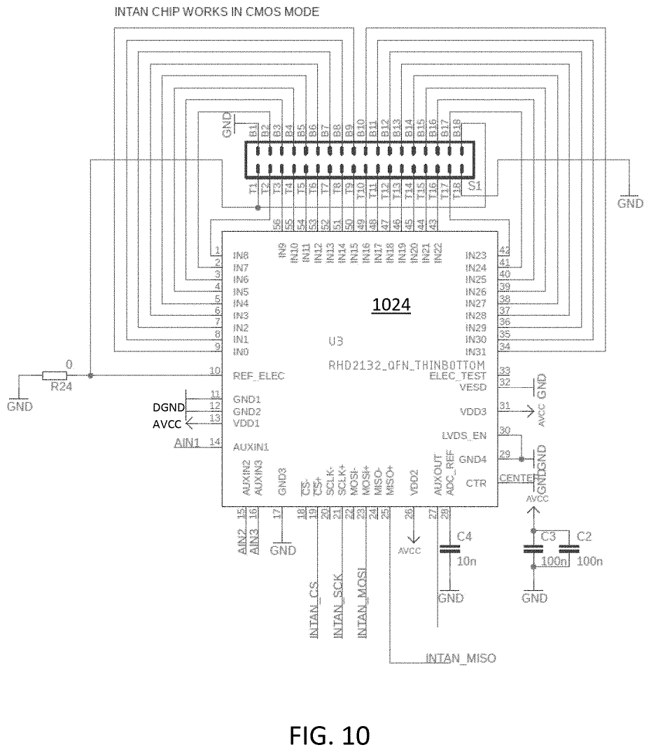

FIG. 10 illustrates an example 1024 of an electrophysiology amplifier that can be used to implement electrophysiology amplifier 924 in accordance with embodiment some embodiments. In some embodiments electrophysiology amplifier 1024 can be model RHD2132 QFN electrophysiology amplifier available from INTAN TECHNOLOGIES of Los Angeles, Calif. Additionally or alternatively, any other suitable electrophysiology amplifier can be used to implement electrophysiology amplifier 924 in some embodiments. As shown in FIG. 10, electrophysiology amplifier 1024 can be connected to a connector S1 for connection to a probe, such as probe 922 of FIG. 1. Probe 922 can be any suitable probe, such as an electrode array for collecting neurological signals from the brain, in some embodiments. During operation, electrophysiology amplifier 1024 can amplify, digitize, and buffer signals from probe 922 and send them to hardware processor 930 via a serial interface (or any other suitable interface), in some embodiments.

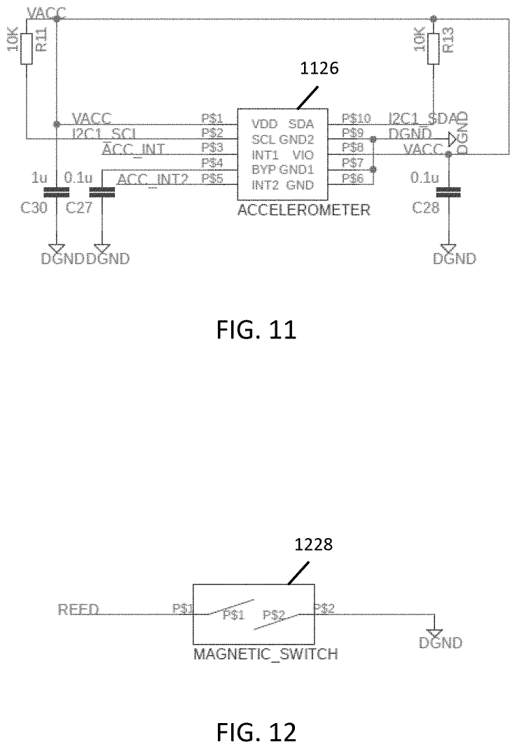

FIG. 11 illustrates an example 1126 of an accelerometer that can be used to implement accelerometer 926, in accordance with embodiment some embodiments. In some embodiments, accelerometer 1126 can be any suitable accelerometer in some embodiments. During operation, accelerometer 1126 can be used to detect motion of a body in which it is located and information regarding the detected motion (such as direction and rate of acceleration) can be provided to hardware processor 930, in some embodiments.

FIG. 12 illustrates an example 1228 of a magnetic switch that can be used to implement magnetic sensor 928, in accordance with embodiment some embodiments. In some embodiments, magnetic switch 1228 can be any suitable magnetic sensor in some embodiments. During operation, magnetic switch 1228, while inside a body, can be used to detecting the presence or absence of a magnet outside the body, and, when the magnet is present, turn transmitter 900 on, present an input to hardware processor 930, and/or perform any other suitable function, in some embodiments.

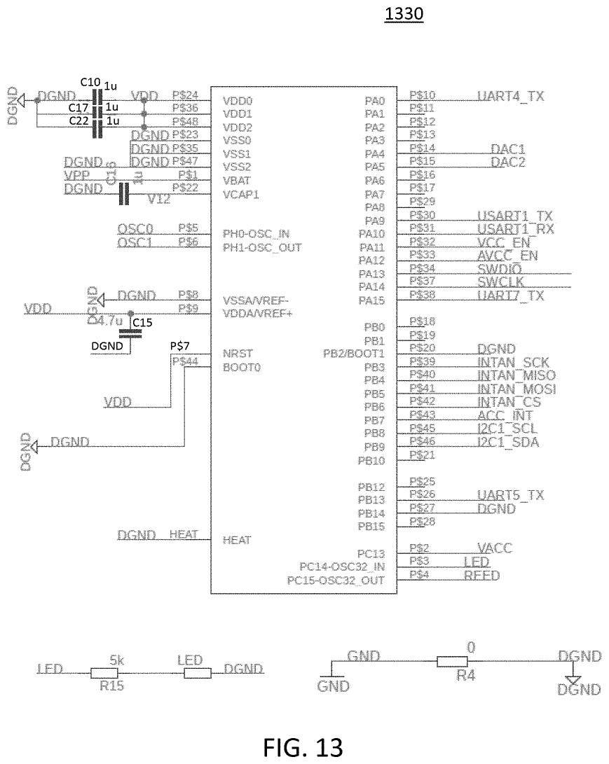

FIG. 13 illustrates an example 1330 of a hardware processor that can be used to implement hardware processor 930, in accordance with embodiment some embodiments. In some embodiments, hardware processor 1330 can be model STM32F413CHU6 available from STMicroelectronics of Plan-les-Ouates, Switzerland. Additionally or alternatively, any other suitable hardware processor can be used to implement hardware processor 920, in some embodiments. During operation, hardware processor 1330 can be used to coordinate data acquisition (e.g., using electrophysiology amplifier 924) process digital data for IC communication, established a serial peripheral interface (SPI) communication, perform UART functions, and/or any other suitable function(s), in some embodiments.

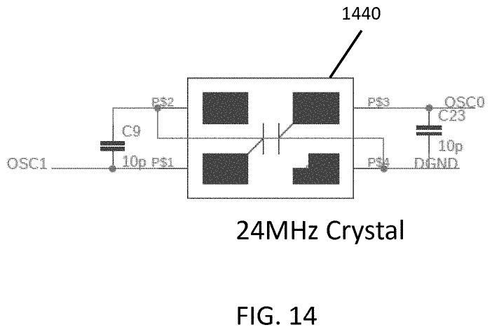

FIG. 14 illustrates an example 1440 of a crystal that can be used to provide oscillator signals to hardware processor 1330 in some embodiments. Any suitable crystal, such as a 24 MHz crystal, can be used in some embodiments.

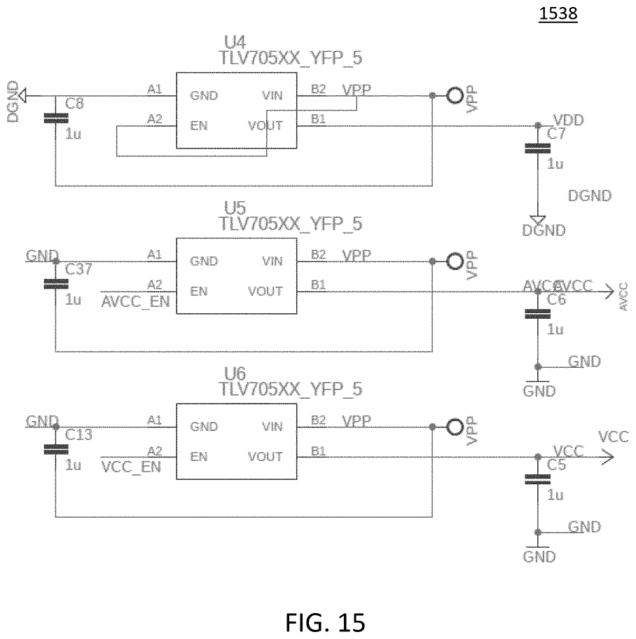

FIG. 15 illustrates examples 1538 of voltage regulators that can be used to implement voltage regulators 938 in some embodiments. In some embodiments, voltage regulators 1538 can be model TLV705 available from Texas Instruments of Dallas, Tex. Additionally or alternatively, any other suitable voltage regulators can be used to implement voltage regulators 938, in some embodiments. During operation, voltage regulators can be used to regulate voltage provided to components of transmitter 900. Although three voltage regulators are shown, any suitable number of voltage regulators, including none, can be used in some embodiments.

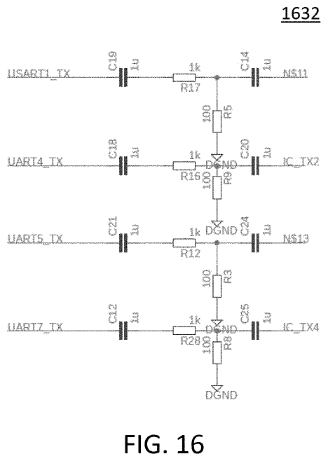

FIG. 16 illustrates an example 1632 of an attenuator and filter that can be used to implement attenuator and filter 932, in accordance with embodiment some embodiments. Additionally or alternatively, any other suitable attenuator and filter can be used to implement attenuator and filter 932, in some embodiments. During operation, attenuator and filter can attenuate and filter transmission signals output by hardware processor 930 so that those signals can be provided to the transmission electrodes, in some embodiments.

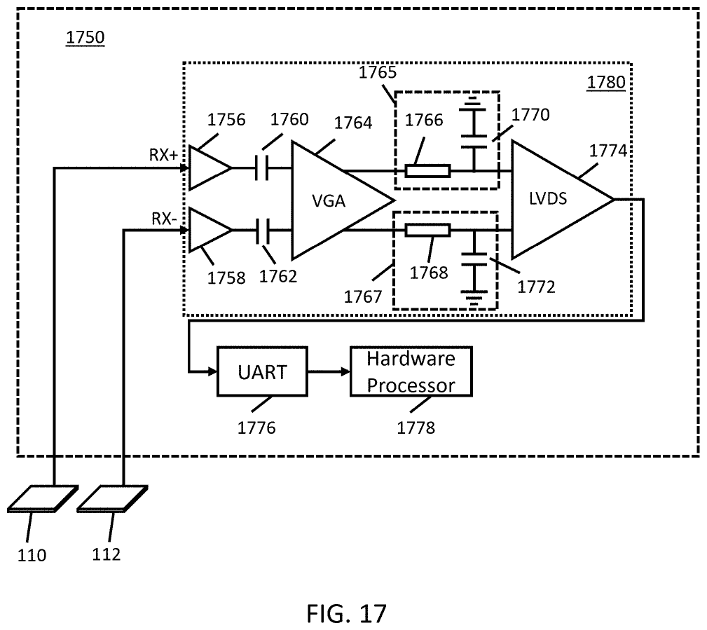

Turning to FIG. 17, an example 1750 of a receiver that can be used to sense receiver electrodes 110,112 of FIG. 1 in accordance with some embodiments is shown. As illustrated, receiver 1750 includes low noise operational amplifiers 1756 and 1758, capacitors 1760 and 1762, variable gain amplifier 1764, filters 1765 and 1767, low-voltage digital signaling (LVDS) receiver 1774, universal asynchronous receiver-transmitter (UART) 1776, and hardware processor 1778. In some embodiments, receiver 1750 can be outside the body of a person or an animal being monitored and positioned so that receiver electrodes 110,112 can be positioned on top of the skin of the person or animal and can receive communications from transmitter electrodes 104 and 106.

In some embodiments, low noise operational amplifiers 1756 and 1758 can be any suitable low noise operational amplifiers, such as model OPA2320 available from Texas Instruments of Dallas Tex. and/or model AD8605 available from Analog Devices of Wilmington, Mass., in some embodiments. During operation, the low noise operational amplifiers can amplify and buffer for the signals received from the receiver electrodes.

In some embodiments, capacitors 1760 and 1762 can be any suitable capacitors that band-pass filter the signal output by the low noise operational amplifiers. The capacitors can band-pass filter at any suitable frequencies, such as 100 kHz-3 MHz, in some embodiments.

In some embodiments, variable gain amplifier 1764 can be any suitable variable gain amplifier, such as model AD8338 available from Analog Devices of Wilmington, Mass., in some embodiments. During operation, the variable gain amplifier can amplify the signal passed by capacitors 1760 and 1762 with automatic gain control.

In some embodiments, filters 1765 and 1767 can be any suitable filters. For example, in some embodiments, filter 1765 can be formed from resistor 1766 and capacitor 1770 and filter 1767 can be formed from resistor 1768 and capacitor 1772.

In some embodiments, low-voltage digital signaling (LVDS) receiver 1774 can be any suitable LVDS receiver, such as model FIN1002 available from ON Semiconductor of Phoenix, Ariz. During operation, the differential output signal of filters 1765 and 1767 is converted by receiver 1774 to logic level.

In some embodiments, universal asynchronous receiver-transmitter (UART) 1776 can be any suitable UART.

In some embodiments, hardware processor 1778 can be any suitable hardware processor, such as model STM32F723ZET available from STMicroelectronics of Plan-les-Ouates, Switzerland. During operation, the hardware processor can receive the signals from the output of UART 1776 and perform any suitable operation on the signals and/or use this signals in any suitable manner.

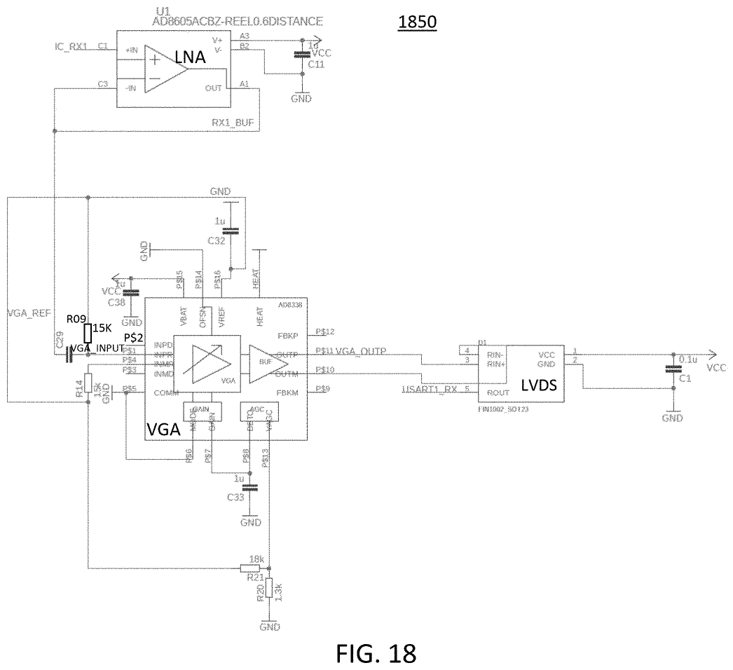

In some embodiments, the components of box 1780 of FIG. 17 can be implemented as shown in circuit 1850 of FIG. 18.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as flash memory, electrically programmable read-only memory (EPROM), electrically erasable programmable read-only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

Although the invention has been described and illustrated in the foregoing illustrative embodiments, it is understood that the present disclosure has been made only by way of example, and that numerous changes in the details of implementation of the invention can be made without departing from the spirit and scope of the invention. Features of the disclosed embodiments can be combined and rearranged in various ways.

What is claimed is:

1. A system for ionic communication in electrolyte, comprising:
a transmitter;
a first plurality of electrodes coupled to the transmitter and in contact with the electrolyte;
a receiver; and
a second plurality of electrodes coupled to the receiver and in contact with the electrolyte,
wherein the transmitter is configured to transmit at least one signal to the receiver by manipulating ions in the electrolyte using the first plurality of electrodes.

2. The system of claim 1, wherein the transmitter and the first plurality of electrodes are configured to be placed inside a body comprising the electrolyte.

3. The system of claim 1, wherein the first plurality of electrodes consists of two electrodes.

4. The system of claim 1, wherein the first plurality of electrodes includes at least three electrodes and the at least one signal is a plurality of signals.

5. The system of claim 1, wherein a voltage of the signal is less than 200 millivolts.

6. The system of claim 1, wherein a frequency of the signal is between 10 kHz to 10 MHz.

8. The system of claim 1, wherein the electrolyte is a human body.

9. The system of claim 1, wherein the at least one signal is transmitted from inside a body to outside the body.

10. The system of claim 1, wherein the first plurality of electrodes includes at least one gold electrode.

11. The system of claim 1, wherein the first plurality of electrodes includes at least one conducting polymer electrode.

12. The system of claim 1, wherein the first plurality of electrodes are arranged in a honeycomb configuration.

13. A system for ionic communication in electrolyte, comprising:
a transmitter; and
a first plurality of electrodes coupled to the transmitter and in contact with the electrolyte;
wherein the transmitter is configured to transmit at least one signal by manipulating ions in the electrolyte using the first plurality of electrodes.

14. The system of claim 13, wherein the transmitter and the first plurality of electrodes are configured to be placed inside a body comprising the electrolyte.

15. The system of claim 13, wherein the first plurality of electrodes consists of two electrodes.

16. The system of claim 13, wherein the first plurality of electrodes includes at least three electrodes and the at least one signal is a plurality of signals.

17. A system for ionic communication in electrolyte, comprising:
a receiver; and
a first plurality of electrodes coupled to the receiver and in contact with the electrolyte,
wherein the receiver is configured to receive at least one signal in response to ions in the electrolyte being manipulated.

18. The system of claim 16, wherein the receiver and the first plurality of electrodes are configured to be placed on top of skin of a body comprising the electrolyte.

19. The system of claim 16, wherein the first plurality of electrodes consists of two electrodes.

20. The system of claim 16, wherein the first plurality of electrodes includes at least three electrodes and the at least one signal is a plurality of signals.
